(19) United States
(12) Patent Application Publication
(10) Pub. No.: US 2025/0134603 A1
(43) Pub. Date: May 1, 2025

(54) ROBOTIC CATHETER SYSTEM AND METHODS FOR DISPLAYING CORRECTIVE ACTIONS THEREFOR

(71) Applicant: CANON U.S.A., INC., MELVILLE, NY (US)

(72) Inventors: Brian Ninni, Woburn, MA (US); Charles George Hwang, Wellesley, MA (US); HuaLei Shelley Zhang, Brookline, MA (US); Fumitaro Masaki, Brookline, MA (US)

(21) Appl. No.: 18/835,295

(22) PCT Filed: Feb. 1, 2023
(86) PCT No.: PCT/US2023/012112

§ 371 Date: Aug. 1, 2024

Related U.S. Application Data
(60) Provisional application No. 63/305,567, filed on Feb. 1, 2022.

(51) Int. Cl. A61B 34/30; A61M 25/01

(52) U.S. Cl. CPC A61B 34/30; A61M 25/0138; A61M 25/0147; A61B 2034/301

(57) ABSTRACT

A robotic catheter system and methods for implementing corrective actions during use thereof. A method comprises: providing a catheter that has one or more bendable segments and a catheter tip, inserting the catheter into a bodily lumen and navigating the catheter through the lumen along an insertion trajectory while an actuation unit applies an actuation force to the one or more bendable segments; recording a navigation parameter in relation to the insertion trajectory while the catheter is moved through the lumen, displaying, on a display, a graphical representation of the navigation parameter relative to a threshold value indicative of a location along the insertion trajectory where a navigation error of the catheter can occur or has occurred; and outputting to the display an interactive user-guide showing one or more than one corrective actions that can be taken to correct and/or prevent the navigation error.

### CROSS-REFERENCE TO RELATED APPLICATIONS

The present application claims priority to U.S. provisional application No. 63/305,567, filed Feb. 1, 2022. The disclosure of the above-listed provisional application is hereby incorporated by reference in its entirety for all purposes. Priority benefit is claimed under 35 U.S.C. § 119(e).

### BACKGROUND INFORMATION

### Field of Disclosure

The present disclosure relates to medical devices. More particularly, the disclosure is directed to robotic catheter systems and methods for displaying information for corrective actions during use thereof.

### Description of Related Art

Robotic catheters or endoscopes include a flexible tubular shaft operated by an actuating force (pulling or pushing force) applied through drive wires controlled by an actuator unit. The flexible tubular shaft (herein referred to as a “steerable catheter”) may include multiple articulated sections configured to continuously bend and turn in a snake-like fashion. Typically, the steerable catheter is inserted through a natural orifice or small incision of a patient's body, and is deployed through a patient's bodily lumen (e.g., an airway or vessel) to a target site, for example, a site within the patient's anatomy designated for an intraluminal procedure, such as an ablation or biopsy. A handheld controller (e.g., a joystick or gamepad controller) may be used as an interface for interaction between the user and the robotic system to control catheter navigation within the patient's body.

The navigation of a steerable catheter can be guided by the live-view of a camera or videoscope arranged at the distal tip of the catheter shaft. To that end, a display device, such as a liquid crystal display (LDC) monitor provided in a console or attached to a wall, displays an image of the camera's field of view (FOV image) to assist the user in navigating the steerable catheter through the patient's anatomy (through a bodily lumen) to reach the target site. The orientation of the camera view, the coordinates of the controller, and the pose or shape of the catheter is mapped (calibrated) before inserting the catheter into the patient's body. As the user manipulates the catheter inside the patient's anatomy, the camera transfers the camera's FOV image to the display device. Ideally, the displayed image should allow the user to relate to the endoscopic image as if the user's own eyes were actually inside the endoscope cavity.

In certain robotic systems, sensors are used for detecting the advancement of the catheter and preventing the robot from causing harm to the patient, for example, by exerting large forces on soft tissue or cutting off blood supply through prolonged pressure exertion and causing ischemia. In that regard, in the current state of the art, there are robotic catheter systems on the market that can keep a record of the forces and/or trajectory of the catheter, and can display a graph of the catheter trajectory along with a history of applied forces, the current pose of the catheter, and even issue warnings to the user. This functionality of a catheter system can allow a user to execute a preventive action, such as stopping movement of the catheter, when a collision of the catheter with the patient's anatomy occurs or is about to occur. See, for example, U.S. Pat. Nos. 9,333,650, 9,770,216, 10,111,723 and 10,271,915, and pre-grant patent application publications US 2022/0125527, US 2020/0054399 and WO 2018/005861. These publications are incorporated by reference herein in their entirety.

While the above-mentioned documents disclose systems for proving indication of the robotic catheter status and warning the operator of catheter collision, no related arts provide an indication for a corrective action to get out from the warning situation or a corrective action to avoid reaching a critical failure situation. For example, while one of the above publications is able to create virtual barriers based on the force measurements at certain positions, the related art does not necessarily guarantee that the user can successfully continue navigation, as it does not address the root cause of the critical situation.

Therefore, there is a need for robotic catheter systems and methods for displaying information to the user in a way to guide the user to execute a corrective action which addresses the root cause of a critical situation.

### SUMMARY OF EXEMPLARY EMBODIMENTS

According to at least one embodiment, it is provided a method of operating a robotic catheter system which is configured to manipulate a steerable catheter having catheter body that includes one or more bendable segments and a catheter tip, and which includes an actuation unit coupled to the bendable segments via one or more drive wires arranged along a wall of the catheter body. The method comprises: inserting at least part of the catheter body into a bodily lumen along an insertion trajectory that follows a lengthwise direction of the lumen; recording a navigation parameter of the catheter while at least the catheter tip is inside the lumen; and displaying a graphical representation of the catheter to show the navigation parameter relative to a threshold value where a navigation error (e.g., catheter collision and/or a system malfunction) can occur or has occurred. An area or volume of the graphical representation shows one or more corrective actions to correct and/or prevent the navigation error.

The various embodiments disclosed in the present disclosure provide several advantages over conventional robotic catheter systems. According to one embodiment, it is advantageous to provide a graphical representation of a force timeline diagram mapping points or events or flags throughout a procedure to forces levels at several thresholds (e.g., each bendable segment of the catheter can have different thresholds). A force timeline diagram with force levels at several thresholds allows the user to resolve navigation errors (catheter collision or system malfunction) by identifying locations where force levels are acceptable (below threshold values), and retracting the catheter tip to such locations. Mapping catheter tip poses throughout the procedure to force levels at certain thresholds, allows the user to avoid future collisions by identifying poses that have potential for minimal collision. A graphical representation of the scale and range of catheter parameters being monitored (wire force, catheter tip location, catheter pose, catheter tip bending angle, etc.) is conveyed to the user in real time. A graphical representation showing different levels of warning allows the user to approach the parameter thresholds with higher precision and confidence to avoid undesired navigation events (catheter collision and/or system malfunction). In particular, when a graphical representation shows navigation parameters associated with conditions of individual drive wires, the user or system can take a corrective action more finely tuned to specific navigation errors.

It is to be understood that both the foregoing summary and the detailed description are exemplary and explanatory in nature and are intended to provide a complete understanding of the present disclosure without limiting the scope of the present disclosure. Additional objects, features, and advantages of the present disclosure will become apparent to those skilled in the art upon reading the following detailed description of exemplary embodiments, when taken in conjunction with the appended drawings, and provided claims.

### BRIEF DESCRIPTION OF DRAWINGS

FIG. 1 illustrates a robotic catheter system 1000 configured to manipulate a steerable catheter 100 having one or more bendable segments and a catheter tip, which can be used in an exemplary medical environment such as an operating room;

FIG. 2 illustrates a functional block diagram and components of the robotic catheter system 1000;

FIG. 3 shows details of a steerable catheter 100 having one or more bendable segments, according to an embodiment of the present disclosure;

FIG. 4A, FIG. 4B, FIG. 4C illustrate various examples of an insertion trajectory for a steerable catheter 100 inserted into a lumen with potential points of catheter collision where a user executes one or more corrective actions, according to an embodiment of the present disclosure;

FIG. 5 illustrates a graphical representation of force timeline diagram associated with an insertion trajectory and a current position of a steerable catheter 100, according to an embodiment of the present disclosure;

FIG. 6 illustrates a graphical representation of a concentric force diagram associated with an endoscope live view image surrounded by a radar plot showing forces applied to the steerable catheter 100 to generate a pose of the catheter tip, according to an embodiment of the present disclosure;

FIG. 7A illustrates an exemplary graph of a generalized catheter parameter x relative to a threshold value where a catheter collision and/or a system malfunction can occur, and FIG. 7B illustrates an exemplary graph of a catheter force parameter relative to a force threshold value where a catheter collision and/or a system malfunction can occur, according to an embodiment of the present disclosure;

FIG. 8 illustrates a graphical user interface for allowing a user to execute corrective actions during navigation of a steerable catheter 100;

FIG. 9A and FIG. 9B illustrate a graphical user interface for allowing a user to execute corrective actions based on parameters of two or more segments of the steerable catheter 100; and

FIG. 10 illustrates a graphical user interface for allowing a user to execute one or more corrective actions to correct or prevent catheter collision and/or system failure, according to an embodiment of the present disclosure; and

FIG. 11 shows an exemplary process 1100 of a method of operating robotic catheter system 1000 for displaying a graphical representation of a navigation parameter relative to a threshold value where a navigation error can occur, wherein a region of the graphical representation shows one or more than one corrective actions that can be taken to correct and/or prevent a navigation error.

### DETAILED DESCRIPTION OF EXEMPLARY EMBODIMENTS

Aspects of the present disclosure can be understood by reading the following detailed description in light of the accompanying figures. It is noted that, in accordance with standard practice, the various features of the drawings are not drawn to scale and do not represent actual components. Several details such as dimensions of the various features may be arbitrarily increased or reduced for ease of illustration. In addition, reference numerals, labels and/or letters are repeated in the various examples to depict similar components and/or functionality. This repetition is for the purpose of simplicity and clarity and does not in itself limit the various embodiments and/or configurations the same components discussed.

Before the various embodiments are described in further detail, it shall be understood that the present disclosure is not limited to any particular embodiment. It is also to be understood that the terminology used herein is for the purpose of describing exemplary embodiments only, and is not intended to be limiting. Embodiments of the present disclosure may have many applications within the field of medical treatment or minimally invasive surgery (MIS).

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise.

As used herein, the term “real-time” is meant to describe processes or events communicated, shown, presented, etc. substantially at the same time as those processes or events actually occur. Real time refers to a level of computer responsiveness that a user senses as sufficiently immediate or that enables the computer to keep up with some external process. For example, in computer technology, the term real-time refers to the actual time during which something takes place and the computer may at least partly process the data in real time (as it comes in). As another example, in signal processing, “real-time” processing relates to a system in which input data is processed within milliseconds so that it is available virtually immediately as feedback, e.g., in a missile guidance, an airline booking system, or the stock market real-time quotes (RTQs).

The present disclosure generally relates to medical devices, and it exemplifies embodiments of an endoscope or catheter, and more particular to a steerable catheter controlled by a medical continuum robot (MCR). The embodiments of the endoscope or catheter and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute. In that regard, all directional references (e.g., upper, lower, upward, downward, left, tight, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure.

As used herein the term “catheter” generally refers to a flexible and thin tubular instrument made of medical grade material designed to be inserted through a narrow opening into an anatomical bodily lumen (e.g., an airway or a vessel) to perform a broad range of medical functions. The more specific term “steerable catheter” refers to a medical instrument comprising an elongated flexible shaft having at least one tool channel spanning through a plurality of bendable segments that are actuated by an actuator that applies an actuation force via drive wires arranged along a wall of the shaft.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

In the present disclosure, the terms “optical fiber”, “fiber optic”, or simply “fiber” refers to an elongated, flexible, light conducting waveguide capable of conducting light from one end to another end due to the effect known as total internal reflection. The terms “light guiding component” or “waveguide” may also refer to, or may have the functionality of, an optical fiber. The term “fiber” may refer to one or more light conducting fibers.

### Robotic Catheter System

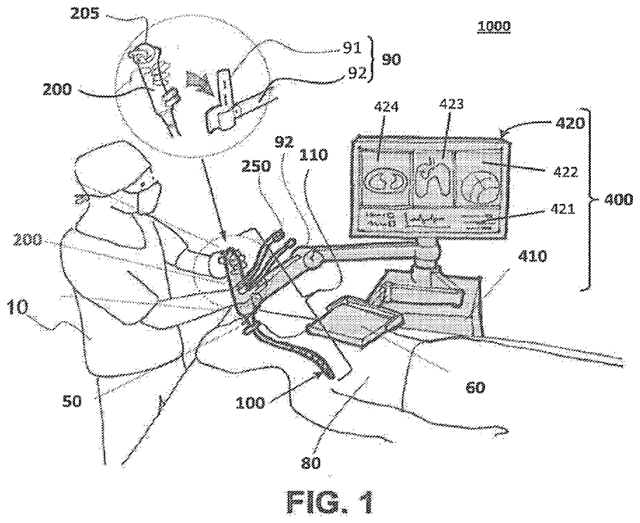

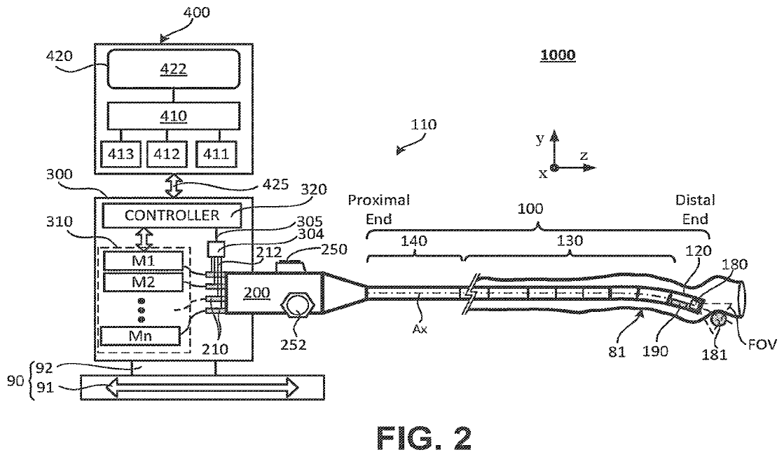

An exemplary configuration of a robotic catheter system 1000 is described with reference to FIG. 1 and FIG. 2. FIG. 1 illustrates an example representation of a medical environment such as an operating room where a robotic catheter system 1000 can be used. The system 1000 may include a robotic catheter 110 operable by a user 10 (e.g., a physician) to perform an intraluminal procedure on a patient 80. The system 1000 may include a computer 400 operatively connected to the robotic catheter 110 via a robotic platform 90. The robotic platform 90 includes one or more than one robotic arm 92 and a translation linear stage 91. The computer 400 (e.g., a system console) includes at least a central processing unit (CPU) 410 comprised of one or more than one processor, and a display screen 420 (display device) such as a liquid crystal display (LCD), OLED or QLED display. As shown in FIG. 2, the CPU 410 is operatively connected to a storage memory 411 (ROM and RAM memory), a system interface 412 (e.g., FPGA card), a user interface 413 (e.g. mouse and keyboard), and to the display screen 420.

The robotic catheter 110 includes a handle 200 and a steerable catheter 100. The steerable catheter 100 is removably attached to the handle 200 via a connector assembly 50 (connector hub). The steerable catheter 100 may also be referred to as a continuum robot catheter or a snake robot catheter configured to form continuous curves based on actuation principles known in the art. A well-known approach to form continuous curves with a continuum robot catheter is the follow-the-leader (FTL) technique. The handle 200 connects to an actuator system 300 which receives electronic commands from the computer 400 to mechanically actuate the steerable catheter 100. The handle 200 is configured to be detachably mounted on the robotic platform 90 for robotically guiding the steerable catheter 100 through a bodily lumen 81 towards a target 181 within the subject or patient 80. When the handle 200 is not mounted on the robotic platform 90, the handle 200 can be operated manually by the user 10 one or more knobs 252 to control the steerable catheter 100. For treating or examining a patient 80, the robotic catheter 110 may include one or more access ports 250 arranged in or around the handle 200. Access ports 250 are used to introduce end effector tools, or to pass fluids to/from the patient 80. A tracking system (comprising, for example, an electromagnetic (EM) field generator 60 and one or more EM sensors 190 arranged on the steerable catheter 100) is used for tracking the position, shape, pose, and/or orientation of the steerable catheter 100 while being inserted through the bodily lumen 81 towards the target 181. The target 181 is a region of interest (e.g., center of a tumor or a lesion) located in or around the lumen 81 of the patient 80. Alternatively or in addition to EM components, the tracking system may include magnetic and/or radiopaque markers.

During an intraluminal procedure, the system's processor or CPU 410 is configured to perform operations based on the user's input by executing (processing) computer-executable code pre-stored in the system's memory 411. The display screen 420 may include a graphical user interface (GUI) configured to display a graphical representation 421 of catheter navigation parameters and patient information, an endoscope image 422 (live view image), an intra-operative guiding image 423, and a pre-operative image 424 (e.g., a 3D or 2D slice image) of a region of interest of the patient 80. Intra-operative guiding image 423 may include conventional fluoroscopy images, or acoustic or ultrasound images. Pre-operative image 424 may include 2D or 3D computed tomography (CT) or magnetic resonance imaging (MRI) images.

As shown in FIG. 2, the steerable catheter 100 is comprised of a proximal section 140, a distal section 130, and a rigid catheter tip 120 arranged in this order from the proximal end to the distal end along a catheter axis (Ax). The distal section 130 is a steerable section comprised of a plurality of bendable segments. The proximal section 140 includes a flexible non-steerable tubular shaft which serves to connect the steerable section 130 to the handle 200. At least the catheter tip 120 includes a tracking sensor 190 (e.g., one or more EM sensors), which is tracked by the system 1000 based on an EM field generator 60.

The steerable catheter 100 is controlled by an actuation system comprised of the handle 200, the actuator system 300, the robotic platform 90 and/or a handheld controller 205 (e.g., a gamepad controller or joystick), which are in electronic communication with the computer 400 via a cable or network connection 425. The actuator system 300 includes a micro-controller 320 and an actuator unit 310 which are operatively connected to the computer 400 via the network connection 425. The micro-controller 320 may include a proportional-integral-derivative (PID) controller or other similar digital signal processor (DSP) circuit. The actuator unit 310 includes a plurality of actuating servo motors (or piezoelectric actuators) M1 through Mn, where “n” can be equal to a number of drive wires 210 necessary for steering the steerable catheter 100.

The robotic control system 300 also includes one or more sensors 304. Sensors 304 can include a strain sensor and/or a position sensor. A strain sensor can be implemented by, for example, a strain gauge or a piezo resistor. A strain sensor serves to detect and/or measure compressive or tensile forces exerted on each drive wire 210. In this case, the strain sensor outputs a signal 305 corresponding to the amount of compressive or tensile force (an amount of strain) being applied to each drive wire 210 during actuation of the steerable catheter 100. The sensors 304 can output a signal 305 corresponding to an amount of movement (distance of displacement) for each actuated drive wire 210. A sensor 304 that measures the amount of displacement of the drive wire can also be implemented by a Hall-effect sensor. The sensor 304 can also be part of a tracking system implemented by an electromagnetic (EM) sensor configured to measure and/or detected the position and orientation (pose) of the catheter tip 120. The signals 305 from the sensors 304 (strain sensor, displacement sensor, and/or pose or position sensor) for one or more drive wires 210 are sent to the controller 320 and/or computer 400 to provide real-time feedback and create closed-loop control for each motor or actuator. In this manner, each drive wire 210 can be actively controlled to implement appropriate shaft guidance for safely navigating the steerable catheter 100 through the lumen 81.

The computer 400 includes suitable software, firmware, and peripheral hardware operated by one or more processor of CPU 410. The computer 400, the actuator system 300, and the handle 200 are operably connected to each other by the network connection 425 (e.g., a cable bundle or wireless link). In addition, the computer 400, the actuator system 300 and the handle 200 are operatively connected to each other by the robot platform 90. In some embodiments, the actuator system 300 may include or be connected to a handheld controller, such as a gamepad controller or a portable computing device like a smart phone or a tablet. Among other functions, the computer 400 and actuator system 300 can provide a surgeon or other operator with a graphical user interface (GUI) and navigation information through the display screen 420 to operate the steerable catheter 100.

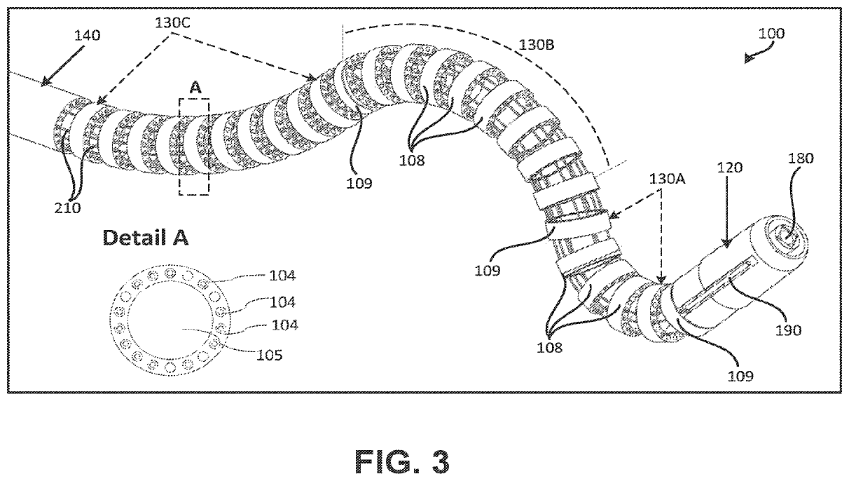

FIG. 3 shows an example embodiment of steerable catheter 100. The proximal section 140 is configured to be attached to the handle 200 via the connector assembly 50. The steerable distal section 130 includes a plurality of bendable segments configured to be actuated by drive wires 210 arranged along the wall of the catheter. The bendable segments of the steerable catheter 100 may include a distal bendable segment 130A, a middle bendable segment 130B, and a proximal bendable segment 130C. Each bendable segment is formed by a plurality of ring-shaped components (rings). The ring-shaped components are defined as wire-guiding members 108 or anchor members 109 depending on their function within the catheter. Anchor members 109 are ring-shaped components onto which the distal end of one or more drive wires 210 are attached. Wire-guiding members 108 are ring-shaped components through which some drive wires 210 slide through (without being attached thereto).

Detail A in FIG. 3 illustrates an exemplary embodiment of a ring-shaped component (a wire-guiding member 108 or an anchor member 109). Each ring-shaped component includes a central opening which forms the tool channel 105, and plural conduits 104 (sub-channels, or thru-holes) formed lengthwise and arranged equidistant from the central opening along the annular wall of each ring-shaped component. The non-steerable proximal section 140 is a tubular shaft made of extruded polymer material. The tubular shaft of the proximal section 140 also has a central opening or tool channel 105 and plural conduits 104 along the wall of the shaft surrounding the tool channel. In this manner, at least one tool channel 105 formed inside the steerable catheter 100 provides passage for an imaging device 180 and/or end effector tools from the access ports 250 to the distal end of the catheter.

An imaging device 180 that can be inserted through the tool channel includes an endoscope camera (videoscope) along with illumination optics (e.g., optical fibers or LEDs). The illumination optics provides illumination light to irradiate a lesion target 181 which is a region of interest within the patient. End effector tools refer endoscopic surgical tools including clamps, graspers, scissors, staplers, ablation or biopsy needles, and other similar tools, which serve to manipulate body parts (organs or tumorous tissue) during examination or surgery.

Next, an example of robotic navigation of the steerable catheter 100 is explained. In general, either during insertion or retraction of the steerable catheter 100 through a bodily lumen 81, the center line of the lumen (e.g., the center line of a lung's airway) is considered the desired trajectory to be followed during active control of the bendable segments of the steerable section 130 (refer to FIG. 3). To that end, various kinematic techniques are used to robotically operate the steerable catheter 100 with the goal of controlling the bendable segments to guide the catheter tip 120 along the desired trajectory to reach a target. In one such example, during robotic navigation, the steerable catheter 100 is advanced through a lumen 81 while sensors measure the insertion depth of the catheter shaft, monitor the force applied to the catheter, and measure angulations of the catheter tip to obtain trajectory information. The trajectory information is stored in a memory of the system and continuously updated. After a short advance in insertion depth, the shape of the steerable catheter is updated by steering (rotating, twisting, or bending) one or more of the bendable segments of the catheter in such a way that the new shape closely matches the desired trajectory. This process is repeated until the target is reached. The same process (in reverse) is applied when the steerable catheter is withdrawn from the patient's lumen. The segments of the distal steerable section 130 can be controlled individually to direct the catheter tip 120 with a combined actuation of all bendable segments, or the catheter tip can be operated in a follow-the-leader (FTL) approach by controlling the most distal segment and the remaining segments following the path traced out by the most distal segment. To withdraw the catheter, a reverse FTL (rFTL) process can be implemented.

In a robotic catheter system as described above, catheter collision with the patient's anatomy can occur when the catheter trajectory is not maintained within the constraints of the lumen. In general, when navigating along a straight section of a lumen, it is desirable to maintain the catheter along the center line of the lumen. If making a turn, the trajectory should be offset away from the center line to navigate “around the corner” of a tight curve, in particular when the catheter has a rigid catheter tip. Therefore, when navigating through tortuous anatomies, the approach path that was followed by the catheter tip can deviate from the intended trajectory for various reasons (e.g., patient movement, user intervention, or the like). User-guided deviation from an ideal path (insertion trajectory) can propagate from the distal most section to the subsequent bendable segments based on the control algorithm used. In addition, the position of the robotically controlled bendable segments can deviate from the user-guided path for numerous reasons, including different section designs, different tolerances for different bendable segments, different positions of the base of that section, etc.

This challenge can frequently arise during navigation due to a number of reasons, as described in the documents disclosed by the related art. A first solution to improve catheter navigation is to address the root cause of what causes errors in the navigation. In other words, according to the present disclosure, one solution is to retract the catheter by a certain amount of the insertion trajectory and try an alternative trajectory approach taking into account a force timeline of the catheter.

### Associating Navigation Force with Timeline of Catheter Position

A first step to be able to retract the catheter and start a new catheter approach is to associate the force applied to the different sections of the catheter with the insertion trajectory that was followed by the catheter during initial insertion. More particularly, the present disclosure proposes to associate the navigation force timeline with the current (live) position of the catheter tip 120 to implement a corrective action when a given error occurs or is about to occur. In this regard, it is known that force-time graphs are known to be used in the state of art to track the position of a steerable catheter, see for example the patents and patent publications listed in the Background section. However, conventional force-time graphs do not provide enough information for the user to take a corrective action that can resolve the root cause of navigation errors (e.g., catheter collision, system malfunction, catheter damage, etc.).

According to an embodiment of the present disclosure, by associating (e.g., mapping or registering) the current (real time) insertion position and pose of the catheter to a timeline of the force or forces (force timeline) applied to the catheter, the system can continuously monitor movement of the catheter and provide actionable feedback in real time. In this manner, the user can immediately understand how the forces imparted onto the catheter tip 120 have changed based on the history of recorded points/locations and corresponding forces applied to the catheter, while it moves through the patient's anatomy. In other words, the system can instruct the user, for example, to retract the catheter to a position/point where an error or collision was not existent or to a point where the catheter was within a predetermined range of safe force values. After the catheter is returned to a position of minimum interference or a position of no collision, the system will prompt the user to repeat insertion with a different (new) trajectory approach. The different trajectory approach can be calculated by the system based on the navigation force timeline that was recorded immediately prior to a given error. Alternatively, the system can calculate a different (new) trajectory based on a difference between a planned insertion trajectory and the trajectory recorded immediately prior to occurrence of a navigation error.

Here, some examples of the occurrence of a navigation error include catheter collisions or system malfunctions of the type known in the art (see for example U.S. Pat. No. 10,111,723). However, the various embodiments disclosed herein are equally applicable any other errors associated with catheter navigation. As long as the system can provide a displayed user-guide that at a glance will enable a user to ascertain the forces in one or more of the drive wires relative to the root cause of the error (a collision, malfunction, damage threshold, etc.), it will be appreciated by those skilled in the art that there is substantial utility in being able to ascertain the wire forces and interactively implement a corrective action. In particular, it is advantageous to ascertain in real-time wire forces relative to the position/orientation of the catheter tip with respect to the lumen, or wire forces relative to each other of the drive wires. For example, if most of the virtual representations of wire forces are close to a threshold value (i.e., close to a maximum tensile or compressive force), the system can provide a useful early warning to the user indicating that something is possibly going wrong.

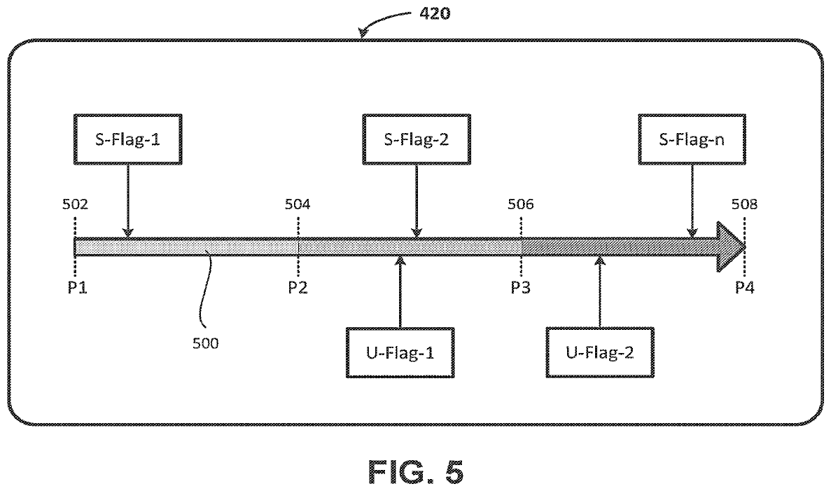

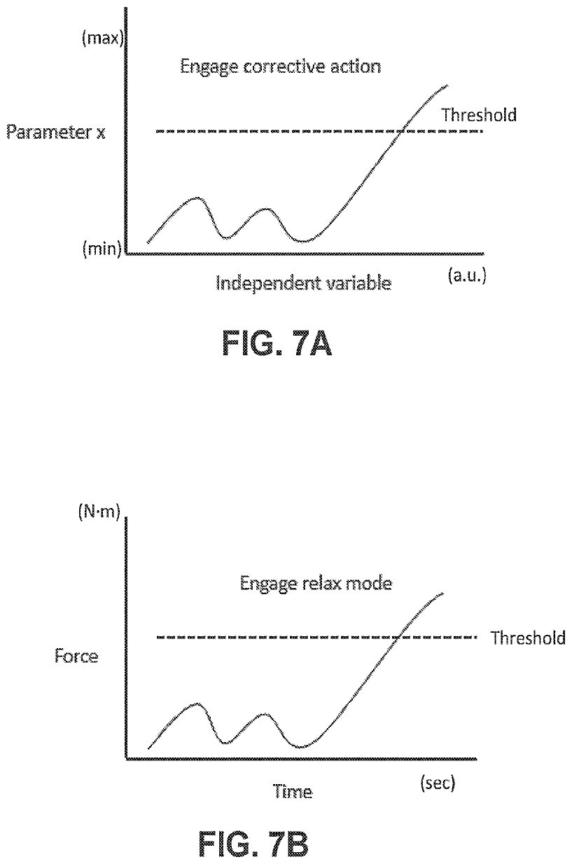

According to one embodiment, the system uses an FTL approach for navigation, and records and displays the history of force or forces (Historical Force Timeline) applied to the different segments of the catheter in association with the insertion trajectory to thereby monitor the current position where a navigation error, such as a collision, occurs or is about to occur. One way this mapping can be displayed to the user is through a force timeline (force-time graph), e.g., as shown in FIG. 5 and FIG. 7B. Advantageously, in addition to the force-time graph, the system provides a user-guide with specific corrective actions to address the root cause of the navigation error.

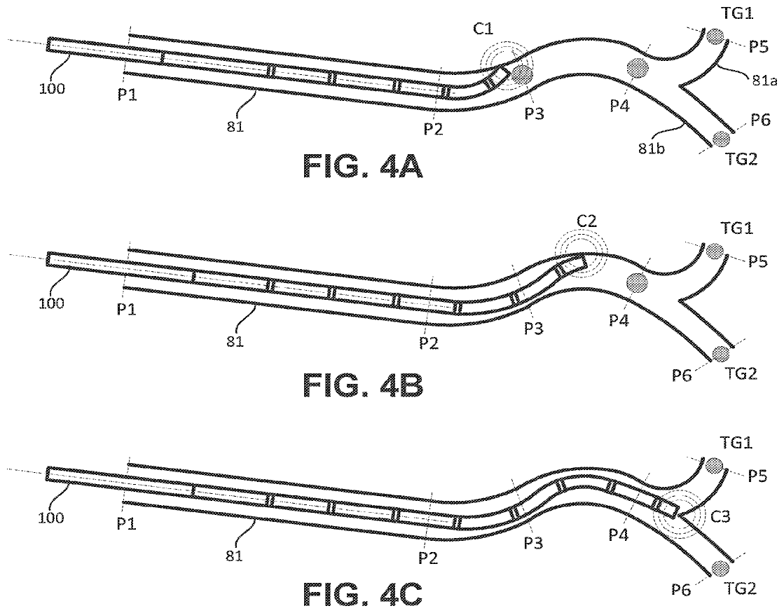

FIG. 4A through 4C graphically illustrates a hypothetical example where a steerable catheter 100 is inserted into lumen 81, and corrective actions are taken by the user to address catheter collision with the lumen. The system can record all three parameters of force, time and position. In one embodiment, the force data is displayed relative to a threshold value along the insertion trajectory. In FIG. 4A, the steerable catheter 100 is initially inserted at a first position P1 and advanced by the linear stage 91 in a linear direction along the center of lumen 81 to a second position P2. After position P2, the robot system controls one or more bendable segments of the steerable catheter 100 to bend one or more segments of the catheter and steer the catheter tip 120 towards a third position P3. To advance to position P3, both the linear stage 91 and one or more bendable segments of the catheter are actuated. When the trajectory taken by the catheter tip is not correct (e.g., when the catheter is bent too much in one direction), the catheter tip 120 may undergo a first collision C1 at a location between the second position P2 and the third position P3. In this case, a solution to guide the catheter tip to the correct trajectory (e.g., center of lumen) is to calculate inverse kinematic values and retract the catheter tip to position P2 (the last known safe position), and then try again. Another solution is to store the force or forces applied to the drive wires for the pose of the catheter at the location of collision C1, then place the catheter in relaxed mode (i.e., stop actuating forces), and automatically retract the catheter until the catheter tip is returned to position P2. At position P2, the system recalculates forward kinematic values for guiding the distal tip 120 to position P3 (“around the corner” of the first curve of lumen 81). This time, the system can use the recorded forces or location coordinates of collision C1 to calculate a new/different trajectory to arrive at position P3 and continue forward.

FIG. 4B illustrates a case where the catheter tip 120 undergoes a second collision C2 between the third position P3 and a fourth position P4. Similar to addressing the previous collision C1, a solution for addressing the second collision C2 is to retract the catheter tip to a safe position (an error-free position) where the catheter was before the collision C2. For example, the catheter tip 120 can be retracted to position P3 by using the system's previously recorded trajectory. At position P3, forward kinematic values are again calculated by the system to maintain the catheter tip 120 along a correct trajectory inside the lumen 81 until the catheter tip reaches the fourth position P4.

FIG. 4C illustrates a case where the catheter tip 120 undergoes a third collision C3 after having passed position P4, and before reaching a first target TG1 at position P5 or a second target TG2 at position P6. In the case where the catheter tip 120 is to be navigated to the second target TG2 through a sub-lumen 81b, catheter tip 120 could be slightly deflected from collision C3 without needing to be retracted backwards. This solution would be adequate because the second target TG2 is already almost aligned with the distal tip 120 of the steerable catheter 100. On the other hand, in the case where the catheter tip 120 is to be navigated to the first target TG1 through a sub-lumen 81a, the catheter tip 120 needs to be retracted backwards to at least position P4 (last known error-free or safe position). After the catheter tip has been retracted to position P4, the system will recalculate the forward kinematics of the catheter to maintain the catheter tip along an approximate center of the sub-lumen 81a until the catheter tip reaches the first target TG1 without further collisions. This process is achieved by continuously monitoring movement of the catheter and providing actionable feedback in real time.

Here, it should be appreciated that collisions of the catheter with the lumen can occur not only as the catheter tip advances through tortuous sections of the lumen, but also as the one or more of the bendable segments become stuck against the lumen wall. In the case where the catheter body becomes stuck against the lumen wall, the system causes the catheter to enter the relaxed mode, retracts the catheter tip to the last known safe position (error-free position), and recalculates the navigation trajectory from the last known safe position to the target. In one embodiment, the system can provide a segmented model of the lung, and display the force data on the segmented model, such that the applied force can be displayed relative to each bifurcation (each carina of the lung airway) along the insertion trajectory of catheter tip. When a corrective action is necessary (e.g., relaxed mode is implemented), the system can record in the force history timeline the insertion depth and catheter pose at the location where the corrective action took place. Thereafter, the system can use the recorded data to return the catheter tip to the latest known error-free position, and/or to avoid navigation through an error-prone position.

FIG. 5 illustrates an embodiment of a graphical representation of a force timeline 500 displayed by the robotic system 1000 on the display screen 420. In this embodiment, the left to right direction of the drawing represents the insertion depth, while the color boundary of each section along the timeline represents the maximum force that the tip experiences at that insertion location. In FIG. 5, the graphical representation of a force timeline 500 includes a plurality of relevant points or tick marks 502-508 to represent navigation events along the insertion trajectory of the steerable catheter 100. For example, a first tick mark 502 on the historic force timeline 500 represents the initial point or start position P1 of the catheter tip insertion into patient's body. The end-point or last tick mark 508 represents the current position (live position) of the catheter tip during a procedure. The intermediate tick marks (second tick mark 504, and third tick mark 506) represent navigation events between the start position P1 and the current position P4 along the catheter trajectory (insertion depth). In some embodiments, the historic force timeline 500 can be displayed in specific colors or patterns, where each color or pattern can be indicative of a distinct navigation event controlled by a separate threshold. For example, the force timeline 500 can be color-coded such that a Green color represents a catheter trajectory where no errors occur; Yellow or Orange color can represent a catheter trajectory where a catheter parameter is close to a threshold value (i.e., a range of force values applied with caution); and Red or blinking Red can represent an event where a catheter collision is imminent or has occurred.

In FIG. 5, the force timeline 500 can be enhanced by adding one or more flags representing certain navigation parameters of the catheter as the catheter advances through the lumen 81. During catheter insertion, one or more flags can be added automatically by the system or manually by the user. During catheter insertion, the user can interact with the force timeline 500 and/or with the flags to see details about the state/condition of the catheter parameter at any point along the history of the insertion trajectory. In FIG. 5, for example, the system automatically adds a first flag (S-Flag-1) at the time when the catheter is inserted into the patient's bodily lumen. S-Flag-1 can be associated with the initial orientation of the catheter tip 120 (e.g., given by the EM sensor 190), the speed of the linear stage 91, etc. The system may also add a second flag (S-Flag-2) when the catheter tip 120 undergoes a first collision C1, and add a third flag (S-Flag-3) when the catheter tip 120 undergoes a second collision C2, etc. Similarly, the user can manually add a first user flag (U-Flag-1) and a second flag (U-Flag-2) to, for example, record the amounts of force that caused a collision or the force that caused the system to change color of the force timeline 500. The flags added by user and/or the system can also be related to number of lumen bifurcations passed by the catheter tip 120 during insertion.

The flags added by the user or the system are not limited to the examples described above. Other flags can be added to record or show other parameters including but not limited to: force values applied at specific points along the insertion trajectory (e.g., force values used to navigate the catheter tip through tortuous curves or bifurcations of the lumen); stage position or stage speed when a catheter collision occurs; EM sensor position/orientation when an error occurs; orientation of endoscope view (e.g., a picture of the live view can be recorded as part of a flag); Catheter Pose (angle and orientation during a collision); Force difference between current force applied and next threshold level, EM Position (or derivative thereof, like estimated insertion amount), percent advanced along a pre-planned insertion trajectory, number of bifurcations passed (e.g., each flag can mark a bifurcation within the lumen), etc.

In one embodiment, the end point of the force timeline 500 can be indicative of 100% of the planned or expected catheter insertion trajectory, and the ‘current’ position will move along the timeline indicating the percentage of insertion as the catheter is moved (inserted or retracted) with respect to the lumen. The percent of insertion along the catheter trajectory can be calculated by, for example, dividing the insertion amount (or estimated insertion amount) by the total path length, and/or by projecting the position of the EM sensor 190 onto the planned trajectory, and calculating the position of the projected EM sensor 190 along the full length of the catheter trajectory.

Some alternative force values for the color-coded sections or points/flags of the force timeline 500 can be: an aggregate of all forces applied to the driving wires 210 of each bendable segment; derived external forces; maximum force encountered at a collision position (rather than the threshold level) where the collision occurred. Derived external forces include the forces experienced by the catheter tip, and which are exerted by external tissues (e.g., due to organ movement). In particular when a catheter does not have a dedicated force sensor at the catheter tip, external forces acting on the catheter are “derived” through known empirical equations using drive wire forces (for example forces sensed by sensors 304). One example of deriving external forces could include the following method: 1. As a calibration process, record the expected forces the drive wires experience when bending a specific pose (in air, with no collision). 2. During the procedure, the system records the forces the drive wires experience during actual catheter insertion. 3. Calculating the difference between 1 and 2 should correlate to the forces being applied to the catheter externally (i.e., from anatomical collision of tissue against the catheter tip). As another example, the force history timeline can be used to determine system malfunction or catheter damage. To that end, the force history timeline can also track the operational force limit of the motor or motors operating the drive wires to give the user an idea of how close to maximum pull/push force the motors are at. This can serve as a sort of a proxy for determining the maximum bend possible. This feature can be important to monitor an inadvertent catheter bend that does not necessarily result in system malfunction, or catheter collision with patient anatomy, or catheter damage. If an inadvertent catheter bend is extreme (e.g., above a given threshold), it can be detected by the force sensors (e.g., sensors 304 shown in FIG. 2). Catheter bending and possible collision events could also be detected using the motor encoders, or other sensor like EM or shape sensors. Shape sensing and shape reconstruction techniques for continuum robots can be done through the utilization of fiber Bragg gratings (FBG) sensing, electromagnetic (EM) tracking or intraoperative imaging. If an extreme bend is detected and no navigation error is determined, the system can still store the position of catheter tip along the force timeline.

In addition, based on user preference, the number of degrees of the color scale for the force timeline 500 can range in discrete values to represent specific colors, or the entire force timeline 500 (or sections thereof) can be displayed in grayscale (shades of gray) with values ranging from, for example, white representing no force (zero force) and black representing maximum force allowed to be applied to the catheter. The force timeline 500 can also be displayed in many alternative ways, besides a color-coded or grayscale one-dimensional timeline.

For example, the force timeline 500 can be alternatively or simultaneously displayed as a Table or List of values mapped with points of forces that fall below (or go above) a certain threshold; as 3D map (e.g., a 3D map where the x-axis is time, the y-axis is the number of drive wires, and the z-axis is the driving wire force). The force timeline in any format can display values, either with the true position of the EM sensor, or with the projected position of EM sensor along a pre-planned trajectory. Instead of using a color to represent force, different force values can be displayed in other ways, such as size/shape (for example when a force of each wire is represented by a dot, the size of dots can be correlated to the amount of force being applied to each drive wire; the same can be done if drive wire force is represented by bars along a linear force timeline). The size/shape would be shown as glyphs in a 3D map. For example, a small sphere could represent small forces, and a large sphere indicates higher forces. Similarly, something like a regular sphere could show no navigation error, while a star with N points could indicate N number of navigation errors (collisions of catheter tip, one or more bending segments getting stuck in the anatomy, etc.).

Once the force timeline is established with recorded points/flags/colors, the user can interact with the force timeline 500 by interactively touching (e.g., with a mouse pointer or manually) these points/flags/colors, etc., to return the catheter tip to the desired position. For example, the user can select a point along the timeline 500, and the system can cause the linear stage 91 to automatically begin to retract the catheter to a safe positon. At the same time, the actuator system will adjust the catheter's pose using inverse kinematics (e.g., using an rFTL algorithm). This process can be implemented iteratively until the catheter tip reaches the selected point (position) along the trajectory within a lumen.

When the system retracts the catheter to a previous position (retracted position), the “registered” points along the history force timeline 500 that are distally beyond the position where the catheter is now located may be removed from the recorded catheter trajectory as these points are no longer relevant or useful to the user for continuing driving the catheter towards the target. However, in at least some embodiments, the user may choose to store the information of the erroneous trajectory as it might be useful for prediction or future collision prevention calculations. In particular, since the discarded information contains data about positions/pose combinations that are known to cause navigation errors, the system may use this information to improve catheter guidance in subsequent insertions or future interventions, where the system can avoid navigation of the catheter through locations (coordinates) or poses known to cause errors.

### Associating Navigation Force with Historical Force Radar Plot

While the above embodiment addresses issues navigation errors resulting from insertion along an insertion trajectory, collisions can also come from the bending motion of the catheter tip without advancing along the insertion trajectory. In fact, collisions during bending can lead to collisions during insertion if the user advances (inserts) the catheter while there is significant collision present. Collisions during bending a distal section can propagate to prior (proximal) sections with FTL control, which also could cause the latter sections to have even more collision against the anatomy due to the subsequent cross talk. For example, if the catheter tip is bending into the lumen wall and the catheter is pushed forward, the middle section will follow that pose and also start to collide with the wall, causing the tip to exert even more force against the anatomy.

In this scenario, the force values can be mapped to not only to the insertion trajectory, but also to the catheter tip position. Force values applied to the different drive wires that control the catheter tip position can be measured by sensors 304, the pose of the catheter tip can be measured by the EM sensor 190. The measured force values can be presented to the user as a radar plot surrounding the endoscope view to better determine a corrective action to be taken.

### Displaying Measured Parameters of Catheter Tip, and Corrective Actions Therefore

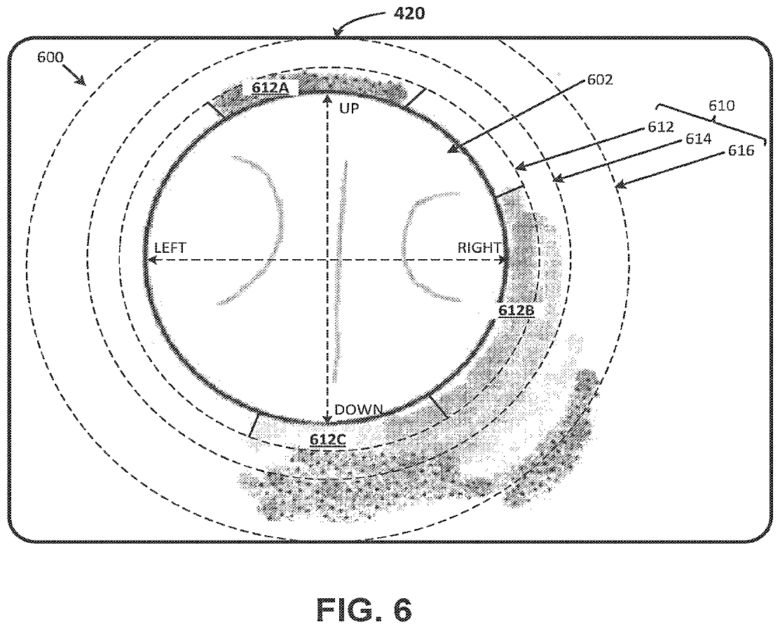

FIG. 6 illustrates a graphical representation 600 of a radar-like force plot 610 for displaying measured parameters of forces applied to generate a pose of the catheter tip 120, according to an embodiment of the present disclosure. In FIG. 6, the center circle represents an endoscope view 602 obtained by the imaging device 180 arranged in the catheter tip 120. Surrounding the endoscope view 602 (in the periphery of the endoscope view 602) is a force plot 610. The force values in the force plot 610 are displayed, and may include a color-coded threshold where the color-coded threshold for the force plot 610 can be similar to the color-coded scheme used for the force timeline 500. In FIG. 6, the lighter portion has a first color as the color of a section 612C, the darker portion has a second color as the color of a section 612B, and the darker portion with small stars has a third color as the color of a section 612A. The endoscope view 602 can display the general orientation (pose) of the catheter tip 120 at a given position (current position) along the insertion trajectory. For example the endoscope view 602 can be mapped with the UP/DOW and LEFT/RIGHT directions in which the catheter tip 120 can be manipulated to reach a desired target location.

The force plot 610 (i.e., the region around the endoscope view 602) indicates the direction (orientation) with respect to the catheter axis where forces were encountered during navigation and bending of the catheter tip in advancing from an insertion point (at coordinates: x=0, y=0, z=0) to the current (live) position. The distance from the endoscope view 602 to the surrounding circle of the force plot 610 can represent a distance from the current position to a positon where the forces were encountered by the catheter tip. For example, a first circle 612 may represent a first distance (e.g., from P4 to P3 in FIG. 4C where the catheter tip is bent and moved within the lumen 81), a second circle 614 may represent a second distance (e.g., P4 to P2) longer than the first distance, and a third circle 616 can represent a third distance (P4 to P1) longer than the sum of first and second distances. Additional circles can be provided from the current position of the catheter tip to the insertion point along the insertion trajectory already traveled by the catheter.

The force values in each circle of distance can be strictly for a specific insertion position (as in embodiment 1) or can represent a navigation error event occurred during catheter insertion. For example, inside the first circle 612, the force values experienced by the catheter tip between the current position (e.g., P4) and the previous position (e.g., P3) are color-coded as a first color in a section 612A, as a second color in a second section 612B, and as a third color in a section 612C. The sections without color inside circle 612 are indicative of zero force being applied in that direction. In the first section 612A, if the force value is represented by a red color, the graphical representation of section 612A in the force plot 610 is indicative of forces above the threshold value causing a possible catheter collision (e.g., C2 in FIG. 4B) or other system malfunction (e.g., excessive force applied to the drive wires actuating the catheter tip in an UP direction). In the second section 612B, if the force value is represented by a green color, the graphical representation of section 612B in the force plot 610 is indicative of a safe operation of the catheter tip below a threshold value. In the third section 612C, if the force value is represented by a yellow or orange color, the graphical representation of section 612C in the force plot 610 is indicative of the force being applied to the catheter tip being near the force threshold value where a navigation error can occur.

The force values can be reset when the stage position moves forward, (or can be re-displayed when the stage position retracts the catheter tip). Alternatively, the force values can be algorithmically expanded to cover multiple points along the insertion trajectory (for example, in increments of +/−5 mm from a given position). These values can be measured by the change in position of the linear stage 91 and the bending of the catheter tip during insertion of the steerable catheter 100 through the lumen 81. The size and range of the circles in force plot 610 can be adjusted to fit the user needs, either manually or automatically.

The graphical representation of the force plot 610 can vary as well (rather than a color-coded threshold), the force plot 610 can be displayed with the same alternatives proposed in the embodiment of FIG. 5. In that regard, the user can interact with the force plot 610 to view additional details about the state of the system when that force was mapped, and the details of corrective action can also be similar to those described in reference to FIG. 5.

The system can also use the information provided by the force plot 610 in multiple ways. For example, the system can completely restrict bending the catheter tip in a given direction when a force in such direction was above a certain threshold. Alternative, the user can select one of the distance circles (e.g., one of circles 612, 614, or 616), and the linear stage 91 can automatically return the catheter tip to the position and pose corresponding to the selected circle. Clicking on a point might not always move the stage, instead the system may be programmed to change the pose of at least one bendable segment of the catheter to match the pose when that force value was recorded (though the system might also move the stage, if the stage position was different when that data point was recorded).

This force plot 610 can be displayed in a few other ways, including, as a pure radar plot without the endoscope view 602 in the center, or as a 3D field in a virtual view. A 3D field in the virtual view could be a series of color-coded points or lines in 3D space (e.g., color coded points or lines superposed on pre-operative CT or MRI 3D images).

The position for each circle in the radar plot 610 around the endoscope view 602 can be calculated in different ways, including tracking the position of the EM sensor 190 along the insertion trajectory, and/or projecting into the current pose's plane the mapped position of the EM sensor 190. In addition, using forward kinematics, the catheter tip 120 can be placed at a position where bending the catheter in a predetermined direction will reach a desired pose. Then, when one of the circles of radar plot 610 is selected by the user, the system can use reverse kinematics to retract the catheter to a predetermined point along the insertion trajectory and to place the catheter tip in the pose/orientation recorded at that position.

### Graphical User Interface for Implementing One or More Corrective Actions

Referring back to FIG. 2 it is recalled that the robotic catheter system 1000 comprises a steerable catheter 100 with plural bendable segments, an actuator unit 310 to bend the bendable segments, and the controller 320 to command the actuator unit 310 based on the operator's input via a joystick or gamepad controller (205 in FIG. 1). The system 1000 also includes a display screen 420 to output the necessary information to the operator 10.

As shown in FIG. 3, each of the three bendable segments of the steerable catheter 100 has a plurality of driving wires 210. If each bendable segment is actuated by three driving wires 210, the steerable catheter 100 has nine driving wires arranged along the wall of the catheter. Each bendable segment of the catheter is bent by the actuator unit 310 by pushing or pulling at least one of these nine drive wires 210. Force is applied to each individual drive wire in order to manipulate/steer the catheter to a desired pose. The actuator unit 310 includes servo-motors to push and pull each of the drive wires 210 individually. Also, the actuator unit 310 has force sensors 304 to measure the forces on the driving wires individually. Since the steerable catheter 100 has operational limits (a bending angle limitation, catheter failure limitation, a limitation of distal force applied by the catheter tip to the anatomy, etc.), the drive wires 210 have an operational limit. This operational limit is stored and/or programmed in the controller 320 or computer 400. Therefore, the controller 320 compares the force measured by the force sensors 304 with the operational limit of each drive wire 210 during the operation.

As the operational limits are approached or reached, the controller warns the operator that a navigation error can occur. In addition to warnings, the present disclosure provides one or more specific corrective actions to be taken by the user. Specifically, for the operational limit on the driving wires, the controller provides a “relax mode”, where the controller performs feedback control with the measured drive wire force values to reduce the force on the drive wires. In the relax mode, the forces applied to the drive wires is reduced such that the steerable catheter 100 can be repositioned to a pose or position which exerts less force on the drive wires. According to at least one embodiment, the controller 320 will interactively guide the operator to execute a corrective action in addition to providing a warning.

According to one embodiment, if we have a generalized parameter X to represent a catheter situation including a critical failure, the controller monitors the parameter X as a function of an independent variable, and compares such parameter with a threshold value. FIG. 7A illustrates an example of a method to display a parameter X versus an independent variable, with a threshold line to indicate when a corrective action should be taken. This threshold value can be the specific value of parameter X or can be a computational value derived from parameter X. It should be understood that parameter X is a catheter parameter that changes dynamically during catheter insertion or catheter bending as a function of, for example, time or distance which the catheter travels through the lumen towards a given target. In other words, the system monitors the catheter parameter X's historical condition relative to an independent variable (other parameters) which can cause navigation errors (e.g., catheter collision, excessive bending force, and/or system malfunction). As the real-time value of parameter X is approaching the threshold value, the system can display this situation with appropriate methods. For example, the controller can display a graphical representation of at least part of the catheter and conditions of parameter X on the display screen 420 to inform the user. When the controller confirms the parameter X reaches the threshold value, the system provides an interactive guide to the operator to execute the corrective action.

In one embodiment, the parameter X can be the measured force on each driving wire 210 during catheter insertion or manipulation (bending or rotating actions), while the corrective action can be an activation of the relax mode (compliant mode) of the steerable catheter 100. FIG. 7B illustrates a method to display a force parameter versus time (force-time graph), with a force threshold to indicate when a corrective action should be taken. According to FIG. 7B, force is on the y-axis of the graph while time is on the x-axis, as force is applied to the catheter's drive wires 210 to manipulate the bending direction (orientation) of the catheter tip 120, time evolves during insertion of the catheter for an intraluminal procedure. The applied force should be maintained below a certain threshold value to prevent catheter collision, system malfunction, and/or to ensure proper movement of the catheter tip without harming the lumen's tissue. If—at certain point in time—the force applied to one or more of the drive wires meets or exceeds the threshold value, the system issues a warning, e.g., a message “engage relax mode” can be shown on the display, and the user or the system implements a corrective action to reduce or release the force applied to the drive wires. According to another example, as the linear stage 91 is controlled to move (insert or withdraw) the catheter through the lumen 81, one or more of force sensors 304 or EM sensor 190 (position sensor) can track the external forces applied to the catheter tip 120 during an intraluminal procedure. If—at certain point in time—the external force applied to the catheter tip exceeds a threshold value, the system issues a warning, e.g., “stop” can be shown on the display, and the user or the system implements a corrective action to, for example, reverse the direction in which the linear stage is driven or to reverse the direction of the force being applied to certain drive wire bending the catheter.

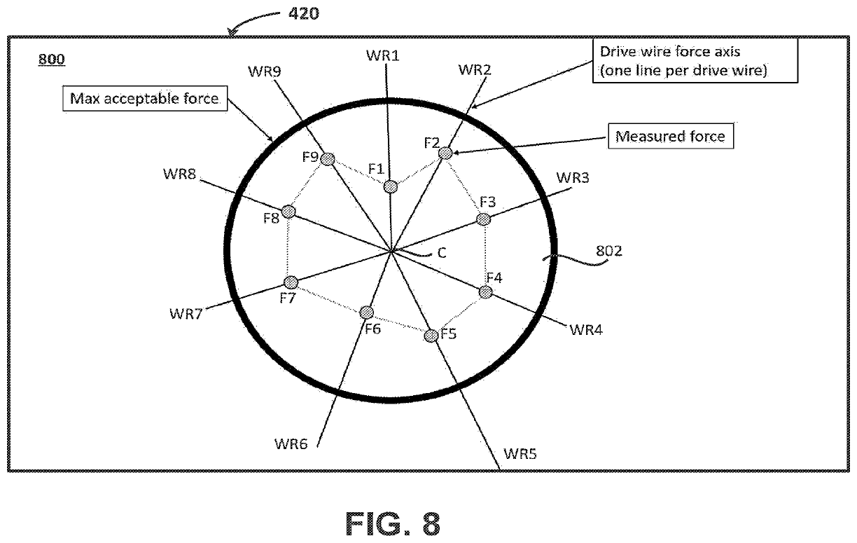

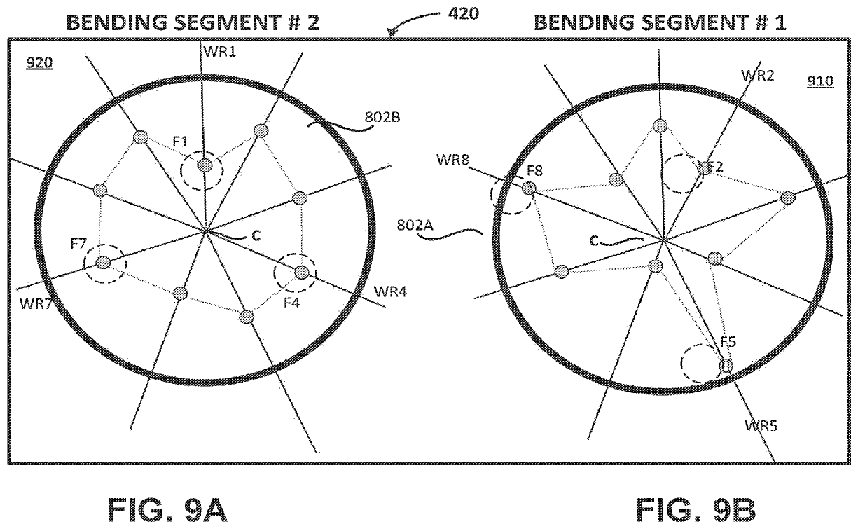

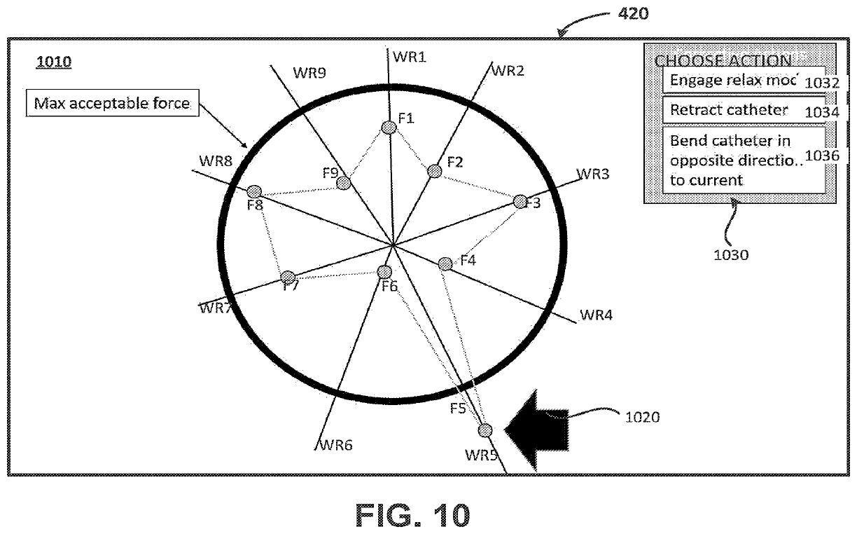

FIG. 8, FIG. 9A-9B, and FIG. 10 illustrate further examples of a graphical representation of the catheter to show a catheter parameter relative to a threshold value where a navigation error (catheter collision, drive wire with excessive force, and/or a system malfunction) can occur or has occurred. FIG. 8 illustrates a graphical representation 800 of monitored drive wire forces shown in display screen 420. According to this embodiment, monitored drive wire forces can be plotted as a spider-web force map. In FIG. 8, a circle 802 represents a maximum acceptable force (a threshold force) to be applied to each of the drive wires 210 of steerable catheter 100. The center C of circle 802 represents the center of the catheter (i.e., the catheter axis Ax). Each line radially extending from the center C to the circle 802 represents an acceptable range of movement of a different drive wire 210. In this example, the spider-web force map includes nine radial lines markers labeled in a clockwise direction as WR1, WR2, WR3, WR4, WR5, WR6, WR7, WR8 and WR9 each representing a range of movement of a drive wire 210. In this graphical representation, the radial direction is indicative of the magnitude of the force applied to each drive wire. The center C of the circle 802 is indicative of zero force applied (i.e., a relaxed mode of the catheter). Dynamic force markers labeled as F1, F2, F3, F4, F5, F6, F7, F8 and F9 respectively arranged on radial lines WR1-WR9 indicate the measured force on that particular drive wire. The ring or circle 802 represents the force threshold that each drive wire should not exceed. When force is applied to any drive wire 210, the corresponding dynamic force marker (F) moves along the radial line (WR); the amount of movement of the force marker F is proportional to the force applied to each wire WR. In the event that the force F applied to one of the drive wires intersects the circle 802 of maximum acceptable force, the system issues a warning to the user. Once any one of the dynamic force markers (F values) enters the zone outside of circle 802, the user should engage a corrective action. Alternatively, the system can be programmed to automatically apply necessary the corrective action. For example, the system can be programmed to automatically enter a relax mode in which actuation force to all wires is stopped.

With this graphical user interface, the controller can inform the user about a situation when the applied force is approaching the threshold value. And, if the applied force becomes equal to or greater than the threshold value, the system can guide the operator to activate the relax mode. Specifically, with this wheel type indication, the system can show the specific direction where the force is increasing and reaching the threshold value. Also, this wheel indication can be aligned to the bending direction of the robotic catheter on the display. For example, when the operator tilts the joystick to upward, the catheter tip 120 of robotic catheter can bend to the upward direction of the display. The same is true for the downward, leftward, and rightward directions. This configuration can provide a preventive action indirectly. For example, when the operator confirms that a high force is being applied in one direction, the operator can understand to bend the robotic catheter to the opposite direction. In addition, by providing a graphical representation of the force applied to each drive wire, the system can more easily determine a system malfunction. For example, if the user confirms that one or more of the force markers (F) do not move along the corresponding axial line (WR), the user can understand that a drive wire may be disconnected or broken or stuck, and therefore not moving.

In other embodiment, the system can have multiple threshold values for the different bendable segments. FIG. 9A and FIG. 9B illustrate an embodiment of a graphical user interface configured to display a spider-web force map for a plurality of bendable segments of the steerable catheter 100. In FIG. 9B, the force map 910 (right side of image) illustrates exemplary force values (navigation parameter) applied to a first bendable segment (e.g., most distal bendable segment), where the force values are limited by a first threshold circle 802A. In FIG. 9A, the force map 920 (left side of image) illustrates exemplary force values (navigation parameter) applied to a second bendable segment (e.g., bendable segment proximal to the most distal bendable segment), where force values are limited by a second threshold circle 802B. It should be understood that a similar graphical representation of navigation parameters and thresholds can be displayed for each bendable segment as a plurality of force maps equal to the number of bendable segments, without limit to the number of bendable segments. Alternatively, force maps for all bendable segments can be combined into a single graphical representation where the drive wires for each bendable segment can be represented radially concentric to each other with respective threshold values displayed therein simultaneously. For example, the graphical representations shown in FIG. 9A and FIG. 9B can be combined into a single display where the most distal bendable segment can be shown in the center, and other bendable segments proximal to the most distal one can be shown radially concentric surrounding the most distal segment, thereby effectively creating a true spider web graphical representation of two or more navigation parameters with respective threshold values thereof.

Under normal operation of the robotic catheter system 1000, as the steerable catheter 100 is advanced through the lumen 81 towards a target 181, forces are applied to the drive wires 210 of each bendable segment to steer the catheter tip 120 in the desired direction. Throughout a typical procedure, the drive wire forces vary, therefore the measured force F on each axial radial line RW will move towards and away from the center C of the force map. For example, in FIG. 9B bendable segment 1 can be actively controlled by 3 drive wires represented by axial line markers WR2, WR5 and WR8; here, when an actuating force is applied to each drive wire, the dynamic force markers F2, F5 and F8 will move accordingly along their respective axial lines. Similarly, in FIG. 9A, bendable segment 2 can be actively controlled by 3 drive wires represented by axial line markers WR1, WR4 and WR7; here, when an actuating force is applied to each drive wire, the dynamic force markers F1, F4 and F7 will move accordingly along their respective axial lines. To display the graphical representation of this embodiment, different colors can be used for representing the drive wires actuated in each bendable segment, and different thresholds can be represented by the circle 802A and circle 802B, respectively. However, to facilitate intuitive understanding that the actuation force is approaching the threshold value, the circle 802A and circle 802B can be drawn with the same color or pattern.

In other embodiments, the data displayed in the spider-web force maps of FIG. 9A and FIG. 9B can represent a difference between the currently measured force being applied to the drive wires and the calculated (of ideal) force for push or pull actuation of the catheter as whole or of individual bendable segments. In a particular embodiment, the calculated (or ideal) force can be used as reference (with assumption of bending the catheter in air) for the push/pull amount of force being applied to the catheter in real time during a procedure. According to this embodiment, by using the force difference between ideal force values and real-time measured force values, the system can calculate the magnitude of external forces acting on the catheter from the interaction between the catheter and the anatomy of the patient, by suppressing the normal operational forces. As a result, the system can highlight more fundamental information relating to the root cause of error or failure. Therefore, the system can provide more intuitive information to the user to more easily implement a corrective action.

FIG. 10 shows a graphical user interface with a graphical representation of navigation parameters displayed as a spider-web force plot 1010 illustrating an error or failure event and corrective actions therefor. According to this embodiment, the system is configured to control a display device to output the spider-web force plot 1010 during an interventional procedure. During normal operational conditions, the force plot 1010 outputs the real time (current) applied force represented by the dynamic movement of one or more force markers F1-F9, as shown in FIG. 8 or FIG. 9. In the event that at least one drive wire 210 is actuated beyond its operational limit and/or the catheter tip undergoes a collision (e.g., collision with sensitive tissue) inside the lumen 81, the system automatically outputs a pop-up window 1030 and a warning indicator 1020. The pop-up window 1030 will provide the user with one or more corrective actions which the user can select, and the waring indicator 1020 will provide indication of the exact location or component the is the root cause of the warning.

For example, as shown in FIG. 10, in case where a drive wire 210 represented by the radial line WR5 is actuated in a manner such that dynamic force marker F5 crosses outside the circle 802, a warning indicator 1020 (an arrow in this example) is prominently shown, and the pop-up window 1030 is immediately shown. Then, the user can choose one or more of a corrective action including, for example, a first corrective action 1032 (“Engage Relax Mode”), a second corrective action 1034 (“Retract Catheter”), a third corrective action 1036 (“Bend catheter in opposite direction”), etc. Once the measured forces are reduced back to within the operational limits, the pop-up window 1030 disappears from the display screen, and normal operation resumes. For example, the system returns to display force plots for each of the bendable segments, as illustrated in FIG. 9A-9B. Here, it should be noted that warning indicator 1020 and pop-up window 1030 corresponds to a region of the graphical representation that shows one or more than one corrective action that can be taken by the user to correct and/or prevent the navigation error of the catheter. In that regard, warning indicator 1020 and pop-up window 1030 applies to all the graphical representations of force history timeline shown in FIG. 4A-4C, FIG. 5, FIG. 7A-7B, and concentric radar force plots shown in FIG. 6, FIG. 8, FIG. 9A-9B, and FIG. 10.

### Software Implementations

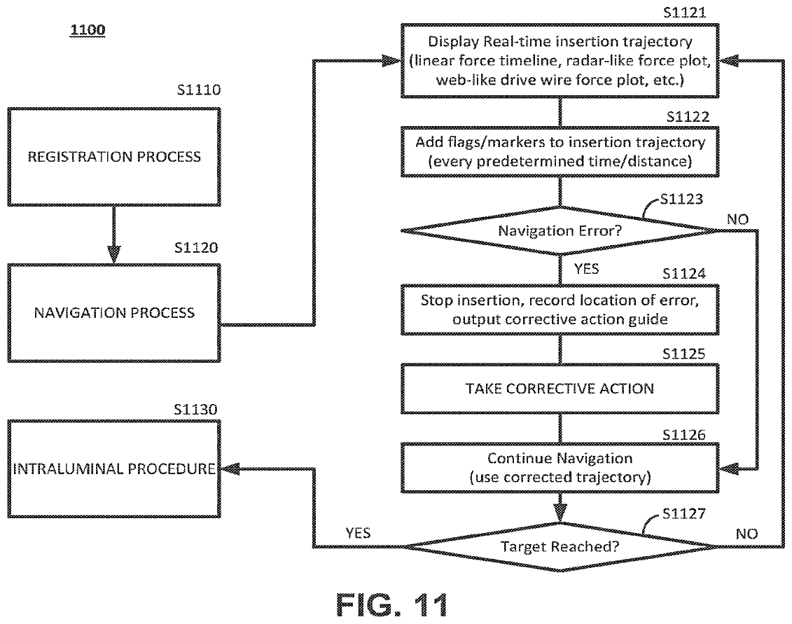

At least certain aspects of the exemplary embodiments described herein can be realized by a computer of a system or apparatus that reads out and executes computer executable instructions (e.g., one or more programs or executable code) recorded on a memory such as a solid state drive (SSD) or an storage medium (which may also be referred to as a ‘non-transitory computer-readable storage medium’) to perform functions of one or more block diagrams, systems, or flowchart described above. FIG. 11 illustrates a flow chart for an exemplary process 1100 of a method of operating a robotic catheter system 1000 which is configured to manipulate a steerable catheter 100 having one or more bendable segments and a catheter tip 120, and which includes an actuator unit 310 (actuator) coupled to the bendable segments via one or more drive wires 210 arranged along a wall of the steerable catheter 100. Process 1100 includes at least a registration process S1110, a navigation process S1120, and an intraluminal procedure S1130.

Registration process S1110 may include catheter-to-patient registration or device-to-image registration where registration of catheter coordinates to coordinates of a tracking system can be performed in any known procedure. Examples of the registration process as described in U.S. Pat. Nos. 10,898,057 and 10,624,701, which are hereby incorporated by reference herein for all purposes.

The navigation process S1120, according to the various embodiments of the present disclosure includes at least the following sub-processes. More specifically, at step S1121, after the steerable catheter 100 has been registered with the patient and/or EM tracking system, the system 1000 enters the navigation process S1120 where the CPU 410 of computer 400 (FIG. 2) continuously records a navigation parameter of the catheter to storage memory 411, and outputs to display screen 420 a graphical representation of the navigation parameter relative to a threshold value indicative of a position where a navigation error of the catheter can occur or has occurred. The navigation parameter relative to a threshold value is shown in real time as graphical representation 421 (in FIG. 1) via the display screen 420. As described above, the navigation parameter can be recorded and displayed as a linear force timeline or a linear force history timeline as shown in FIG. 5. Here, the system automatically performs force-time data collection during catheter insertion. In other embodiments, as shown in FIG. 6 and FIG. 8 through FIG. 10, navigation parameters can be recorded and displayed as concentric force plots representing catheter tip bending pose or representing force levels applied to individual drive wires during actuation. At step S1122, the system and/or the user may add flags or markers to the recorded navigation parameters. Flags or markers can be recorded automatically by the system (e.g., S-Flag-1, S-Flag-2, etc.) or can be manually recorded by the user (e.g., U-Flag-1, U-Flag-2, etc.), as shown in FIG. 5. These flags, markers or points can be added every predetermined time or distance interval (e.g., every few seconds or every few millimeters of catheter insertion).

At step S1123, the system continuously monitors the navigation parameters, and determines whether a navigation error occurs. As explained in detail above, the system continuously compares a navigation parameter to a threshold value to determine if a navigation error (e.g., catheter collision, system malfunction, etc.) has occurred. If a navigation error does not occur (NO at S1123) the process continues to step S1126.

If a navigation error can occur or has occurred (YES at S1123), at step S1124, the system may stop catheter insertion, record the location of the error, and output a guide for corrective action to be taken by the user. For example, a popup window 1030 within the graphical representation of the navigation parameter can provide one or more corrective actions which the user can take at step S1125 to correct the navigation error. At step S1125, the user can take one or more of several corrective actions as described above in reference to FIG. 10. Alternatively, or in addition thereto, the system can be programmed to automatically implement a corrective action, such as making the steerable catheter compliant by entering a relax mode by releasing the actuating forces applied to drive wires.

After the corrective action is taken, at step S1126, the system continues monitoring the navigation of the catheter. In this step, if the action taken by user includes a corrective action 1034 (retract catheter), the system will use a corrected insertion trajectory to advance the catheter. If the action taken by user includes a corrective action 1036 (bend catheter in opposite direction), the system will display the effect of such corrective action. For example, the display screen 420 of in FIG. 10 will show when the force marker F5 returns to the inside of circle of maximum acceptable force. Thereafter, the system will use the corrected insertion trajectory to advance the catheter.

At step S1127, the system determines if the catheter has reached the intended target. If the catheter has not yet been reached (NO in S1127), the process returns to step S1121 where the navigation parameter continues to be monitored in real time. If the catheter has safely reached the intended target (YES at S1127), the process transitions to step S1130, where the user can perform the desired interventional procedure (e.g., ablation, biopsy, or the like).

The computer 400 may include various components known to a person having ordinary skill in the art. For example, the computer may include signal processor implemented by one or more circuits (e.g., a field programmable gate array (FPGA) or an application specific integrated circuit (ASIC)) for performing the functions of one or more of the above-described embodiment(s), by, for example, reading out and executing the computer executable instructions from the storage medium to perform the functions of one or more of the above-described embodiment(s) and/or controlling the one or more circuits to perform the functions of one or more of the above-described embodiment(s). The computer may comprise, as the CPU 410, one or more processors, a micro processing unit (MPU), and may include a network of distributed remote computers or separate processors (cloud computing) to read out and execute the computer executable instructions. The computer executable instructions may be provided to the computer, for example, from a cloud-based network or from the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. The computer may include an input/output (I/O) interface to receive and/or send communication signals (data) to input and output devices, which may include a keyboard, a display, a mouse, a touch screen, touchless interface (e.g., a gesture recognition device), a printing device, an stylus, an optical storage device, a scanner, a microphone, a camera, a network drive, a wired or wireless communication port, etc.

The various embodiments disclosed in the present disclosure provide several advantages over conventional robotic catheter systems. According to one embodiment, it is advantageous to provide a graphical representation of a force timeline diagram mapping points throughout a procedure to force levels at several thresholds (e.g., each bendable segment of the catheter can have a different threshold). For example, the most distal bendable segment can have the most sensitive (e.g., lowest) threshold because the catheter tip may have a higher probability of collision with the patient's lumen. Catheter bendable segments other than the most distal one can have an increasingly less sensitive (e.g., higher) threshold to monitor potential events where a curved section becomes stuck against the lumen's tissue even if the distal tip is correctly positioned. A force timeline diagram with force levels at several thresholds allows the user to resolve navigation errors (e.g., catheter collision or system malfunction) by identifying locations along the insertion trajectory where force levels are acceptable (below threshold values). Mapping catheter tip poses throughout the procedure to force levels at certain thresholds, allows the user to avoid future collisions by identifying poses that have potential for minimal collision. A graphical representation of the scale and range of the catheter parameter being monitored (wire force, insertion depth, catheter tip collision, for instance) is conveyed to the user in real time. A graphical representation showing different levels of warning allows the user to approach the parameter thresholds with higher precision to avoid one or more of catheter collision, system malfunction, system failure, or patient harm. In particular, when a graphical representation shows catheter parameters associated with conditions of individual drive wires, the user or system can take a corrective action more finely tuned to prevent potential system malfunction or patient harm.

The present application also discloses various aspects of a robotic catheter system. According to aspect (1), the robotic catheter system comprises: a catheter having one or more bendable segments and a catheter tip; an actuator coupled to the bendable segments of the catheter via one or more drive wires arranged along a wall of the catheter; a processor in operative communication with the actuator; and a memory storing instructions that, when executed by the processor configures the processor to: continuously record a navigation parameter while the catheter is inserted through a lumen along an insertion trajectory; cause a display device to display a graphical representation of the navigation parameter relative to a threshold value indicative of a position along the insertion trajectory where a navigation error of the catheter can occur or has occurred, wherein a region of the graphical representation shows one or more than one corrective action that can be taken to correct and/or prevent the navigation error of the catheter.

Other aspects of the catheter system according to the present application include instructions that, when executed by the processor further configures the processor to implement an aspect (2) to record, as the navigation parameter, a positional parameter associated with a real-time position or orientation of the catheter tip with respect to the lumen; aspect (3) to control the actuation unit to apply a force to the drive wire to bend at least one the one or more bendable segments of the catheter, wherein the processor records, as the navigation parameter, a force parameter associated with a push or pull force applied by the actuation unit to the one or more drive wires to bend the one or more bendable segments of the catheter; an aspect (4) wherein the processor records, as the navigation parameter, one or more of aspect (4a) a force parameter associated with a position where a catheter collision occurs, aspect (4b) a percentage of distance traveled by the catheter tip along a pre-planned insertion trajectory until a catheter collision occurs, and aspect (4c) a number of lumen bifurcations passed by the catheter tip before a navigation error occurs; aspect (5) wherein the processor records, as the navigation parameter, a linear force timeline associated with the insertion trajectory which was followed by the catheter tip during insertion into the lumen, and the linear force timeline includes force levels of a push or pull force applied by the actuation unit to the one or more drive wires to bend the one or more bendable segments of the catheter; and aspect (6) wherein the processor controls the display device to display one or more markers or flags throughout the linear force timeline, and the one or more markers or flags show force levels that approach or cross the threshold value where a navigation error can occur or has occurred.

Aspect 7: The system according to aspect (6), wherein the processor is further configured to: control the display device to display a guide for the user to take corrective action to resolve the navigation error by identifying at least one of a point or a marker or a flag in the linear force timeline where the force levels are below the threshold value; cause the catheter tip to retract to a retracted position inside the lumen corresponding to the identified at least one of the point or marker or flag in the linear force timeline, and continue to insert the catheter into the lumen from the retracted position using a corrected insertion trajectory.

Aspect 8: The system according to aspect (1), wherein the processor is further configured to record catheter tip positions along the insertion trajectory during insertion of the catheter into the lumen, wherein the recorded catheter tip positions are associated with force levels of a force applied by the actuation unit to the one or more drive wires to bend the catheter tip.

Aspect 9. The system according to aspect (8), wherein the processor is further configured to display, as the graphical representation, a radar-like force plot having one or more circles indicative of the catheter tip positions where the force levels approach or cross the threshold value where the navigation error can occur, and wherein the force levels are displayed in different color-coded values or different patterns in at least one of the one or more circles.

Aspect 10. The system according to aspect 1, wherein the processor is further configured to: control an imaging device arranged inside the catheter tip to acquire a live view image of the lumen; and display, on the display device, the live view image of the lumen together with the graphical representation of the navigation parameter.

Aspect 11. The system according to aspect 10, wherein the processor displays the live view image together with the graphical representation of the navigation parameter includes displaying the live view image surrounded by a radar-like force plot associated with a position and/or orientation of the catheter tip inside the lumen.

Aspect 12. The system according to aspect 10, wherein processor displays, as the live view image together with the graphical representation of the navigation parameter, a radar-like force plot showing forces applied by the actuation unit to the one or more drive wires to bend the catheter tip in one or more directions including an UP direction, a DOWN direction, a LEFT direction, a RIGHT direction, or combinations thereof with respect to the live view image.

Aspect 13. The system according to aspect 1, wherein the processor records, as the navigation parameter, a position of the catheter tip with respect to a wall of the lumen and/or records an orientation of the catheter tip with respect to the catheter axis.

Aspect 14. The system according to aspect 13, wherein the processor displays, as a graphic representation of the navigation parameter, a virtual representation of the one or more drive wires in a radial arrangement, and displays the threshold value as a threshold circle surrounding the radial arrangement of the one or more drive wires.

Aspect 15. The system according to aspect 14, wherein the virtual representation of the one or more drive wires is displayed as a force marker for each drive wire of the steerable catheter, and wherein the each force marker is configured to dynamically move in a radial direction in response an amount of force applied by the actuation unit to the one or more drive wires for bending the catheter tip.

Aspect 16. The system according to aspect 14, wherein the one or more bendable segments include a first bendable segment and a second bendable segment, in order from the distal to the proximal end of the catheter, wherein the processor displays, as a virtual representation of the one or more drive wires, a first radial arrangement of force markers for one or more drive wires of the first bendable segment, and displays a second radial arrangement of force makers for one or more drive wires of the second bendable segment, wherein each of the force markers of the first radial arrangement moves radially in response to the force applied by the actuation unit to bend the first bendable segment, and each of the force markers of the second radial arrangement moves radially in response to the force applied by the actuation unit to bend the second bendable segment.

Aspect 17. The system according to aspect 14, wherein the virtual representation of the one or more drive wires shows each of the one or more drive wires as a force marker which moves radially in response to the force applied by the actuation unit to bend the catheter tip in an UP direction, or a DOWN direction, or a LEFT direction, or a RIGHT direction, or combinations thereof with respect to the catheter axis.

Aspect 18. The system according to aspect 17, wherein, when an amount of force applied by the actuation unit to the one or more drive wires for bending the one or more bendable segments becomes equal to or greater than the threshold value, a portion of the graphical representation shows a popup window listing one or more than one corrective action to correct and/or prevent the navigation error.

Aspect 19. The system according to aspect 1, wherein the processor is further configured to: detect that a navigation error has occurred when a value of the navigation parameter becomes equal to or greater than the threshold value; and take a corrective action to correct the navigation error.

Aspect 20. The system according to aspect 19, wherein the processor is further configured to: record a location along the insertion trajectory where the navigation error has occurred, and display, along with the graphic representation of the navigation parameter, the location along the insertion trajectory where the navigation error has occurred.

Aspect 21. The system according to aspect 20, wherein the processor displays, as the graphic representation of the navigation parameter, a linear force timeline of the insertion trajectory, and wherein the processor, as the location where the navigation error has occurred, automatically records or prompts the user to record a flag or a marker to the linear force timeline, wherein the flag or marker is indicative of the location where the navigation error has occurred.

Aspect 22. The system according to aspect 20, wherein the processor is further configured to: place the catheter in a relaxed mode based on detection that a navigation error; retract the catheter tip to a position along the insertion trajectory proximal to the flag or marker; and navigate the catheter along a corrected insertion trajectory by using an actuation force lower than the threshold value.

### Modifications and/or Combinations of Embodiments

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by persons of ordinary skill in the art to which this disclosure belongs. In that regard, breadth and scope of the present disclosure is not limited by the specification or drawings, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. All embodiments can be modified and/or combined to improve and or simplify the anti-twist feature as applicable to specific applications. The scope of the following claims is to be accorded the broadest interpretation so as to encompass all such modifications and equivalent structures and functions.

Any patent, pre-grant patent publication, or other disclosure, in whole or in part, that is said to be incorporated by reference herein is incorporated only to the extent that the incorporated materials do not conflict with standard definitions or terms, or with statements and descriptions set forth in the present disclosure. As such, and to the extent necessary, the disclosure as explicitly set forth herein supersedes any conflicting material incorporated by reference.

1-45. (canceled)

46. A method for catheter navigation, the catheter including one or more bendable segments actuatable by an actuation via one or more drive wires, the method comprising:
inserting the catheter into a lumen;
navigating the catheter through the lumen along an insertion trajectory;
recording a navigation parameter in relation to the insertion trajectory while the catheter is inserted into the lumen;
displaying, on a display, at least one of the navigation parameter and/or a representation of the navigation parameter indicative of a location along the insertion trajectory where a navigation error can occur or has occurred; and
outputting, to the display, an interactive user-guide of at least one corrective action to correct and/or prevent the navigation error.

47. The method of claim 46, wherein recording the navigation parameter includes at least one of:
recording at least one force parameter associated with a push or pull force applied to the one or more drive wires to bend the one or more bendable segments of the catheter,
recording a real-time position or orientation of a tip of the catheter with respect to the lumen while navigating the catheter through the lumen,
recording at least one force parameter associated with a position where a catheter collision with the lumen occurs,
recording a percentage of distance traveled by the catheter tip along a pre-planned insertion trajectory when a catheter collision occurs,
recording a number of lumen bifurcations passed by the catheter tip before the navigation error occurs,
recording a force timeline associated with the insertion trajectory followed by the catheter tip during insertion into the lumen, and/or
recording positions of catheter tip along the insertion trajectory during insertion of the catheter into the lumen, with the recorded positions being associated with force levels applied to the one or more drive wires.

48. The method of claim 47, wherein:
the force timeline includes at least one of: (a) force levels of at least one of a push force and/or a pull force applied to the one or more drive wires to bend the one or more bendable segments of the catheter, and/or (b) a linear force timeline with one or more markers or flags thereon, and
a threshold value indicates the location where the navigation error can occur or has occurred, and the one or more markers or flags correspond to force levels that approach or cross the threshold value where the navigation error can occur or has occurred.

49. The method of claim 46, further comprising:
displaying a guide to resolve the navigation error by identifying at least one of a point, a marker, and/or a flag in a linear force timeline where levels of the actuation force are below a threshold value;
causing a tip of the catheter to retract to a retracted position inside the lumen corresponding to the identified at least one of the point, the marker, and/or the flag in the linear force timeline;
calculating a corrected insertion trajectory based on the linear force timeline; and
inserting, using the corrected insertion trajectory, the catheter tip into the lumen from the retracted position.

50. The method of claim 46, wherein:
displaying the at least one of the navigation parameter and/or the representation of the navigation parameter includes at least one of: (a) displaying a virtual representation of the one or more drive wires in a radial arrangement, and/or (b) displaying a threshold value as a circle surrounding the radial arrangement of the one or more drive wires,
the virtual representation is displayed as a force marker for each drive wire of the steerable catheter, and
each force marker is configured to at least one of: (a) move radially in response an amount of force applied to the one or more drive wires, and/or (b) move radially in response to the actuation force to bend a tip of the catheter in at least one of an up direction, a down direction, a left direction, and/or a right direction, with respect to an axis of the catheter.

51. The method of claim 50, wherein:
the virtual representation includes a first radial arrangement of force markers for one or more drive wires of a first bendable segment of the one or more bendable segments, and a second radial arrangement of force makers for one or more drive wires of a second bendable segment of the one or more bendable segments, and
each force marker of the first radial arrangement moves radially in response to the actuation force to bend the first bendable segment, and each force marker of the second radial arrangement moves radially in response to the actuation force to bend the second bendable segment.

52. The method of claim 46, wherein recording the navigation parameter includes recording a position of a tip of the catheter with respect to a wall of the lumen and/or recording an orientation of the catheter tip with respect to an axis of the catheter.

53. The method of claim 46, further comprising:
controlling an imaging device arranged inside a tip of the catheter to acquire a live view image of the lumen; and
displaying the live view image of the lumen with the representation of the navigation parameter.

54. The method of claim 53, wherein:
the displayed live view image is surrounded by a radar-like force plot associated with a position and/or orientation of the catheter tip inside the lumen, and
the radar-like force plot corresponds to forces applied to the one or more drive wires to bend the catheter tip in one or more directions including an up direction, a down direction, a left direction, a right direction, or combinations thereof with respect to the live view image.

55. The method of claim 46, wherein, when the actuation force reaches or exceeds a threshold value, at least a portion of the representation of the navigation parameter includes at least one of a warning or a pop-up window listing the at least one corrective action.

56. The method of claim 46, further comprising:
recording a location along the insertion trajectory where the navigation error has occurred, and
displaying a graphical representation of the navigation parameter with the location along the insertion trajectory where the navigation error has occurred.

57. The method of claim 56, wherein:
displaying the graphical representation of the navigation parameter includes displaying a linear force timeline of the insertion trajectory, and
recording the location where the navigation error has occurred includes adding at least one of a flag and/or a marker to the linear force timeline indicative of one or more of: (a) the location where the navigation error has occurred and (b) the amount of the actuation force applied to the drive wires at said location.

58. The method of claim 46, further comprising:
placing the catheter in a relax mode in which the actuation force is not applied to the drive wires;
retracting a tip of the catheter to a position along the insertion trajectory adjacent to the location where the error has occurred; and
navigating the catheter along a corrected insertion trajectory by modifying the actuation force to be equal to or lower than a threshold value.

59. A system, comprising:
a steerable catheter having one or more bendable segments;
an actuator configured to be coupled to the one or more bendable segments via one or more drive wires;
a memory; and
a processor in operative communication with the actuator and the memory, wherein
the memory is configured to store instructions that, when executed by the processor, configure the processor to:
record a navigation parameter during insertion of the catheter through a lumen along an insertion trajectory;
output at least one of the navigation parameter and/or a representation of the navigation parameter relative to a position along the insertion trajectory where a navigation error can occur or has occurred; and
output an interactive user-guide including at least one corrective action for correcting and/or preventing the navigation error.

60. The system of claim 59, wherein, when executed by the processor, the instructions further configure the processor to:
record, as the navigation parameter, a positional parameter associated with a real-time position or orientation a tip of the catheter with respect to the lumen;
control the actuator to apply a force to at least one drive wire of the one or more drive wires to bend at least one segment of the one or more bendable segments of the catheter; and
record, as the navigation parameter, at least one of:
a) a force parameter associated with a push or pull force applied by the actuator to the one or more drive wires to bend the one or more bendable segments of the catheter;
b) at least one force parameter associated with a position where a catheter collision occurs;
c) a percentage of a distance traveled by the catheter tip along a pre-planned insertion trajectory until the catheter collision occurs;
d) a number of lumen bifurcations passed by the catheter tip before the navigation error occurs; and
e) a linear force timeline associated with the insertion trajectory followed by the catheter tip during insertion into the lumen, wherein the linear force timeline includes force levels of a push or pull force applied by the actuator to the one or more drive wires to bend the one or more bendable segments of the catheter.

61. The system of claim 59, wherein, when executed by the processor, the instructions further configure the processor to:
detect that the navigation error has occurred based on the navigation parameter exceeding a threshold value, and
prompt a user to take a corrective action to correct the detected navigation error, with the corrective action being one or more of: (a) entering a relax mode, (b) stopping insertion of the catheter, (c) reversing a direction of movement of the catheter, and/or (d) reversing a direction of a force being applied to at least one drive wire.

62. A system, comprising:
a catheter that has one or more bendable segments and at least one sensor at a distal end thereof; and
a processor configured to:
control insertion of at least a portion of the catheter along an insertion trajectory;
control application of a force to the one or more bendable segments to generate a pose of the catheter;
record force data at a plurality of time-points and/or a plurality of positions along the insertion trajectory using the at least one sensor; and
control display of a representation of the force data relative to at least one of an insertion depth and the pose.

63. The system of claim 62, wherein the processor is further configured to:
control display of, at least one flagged point based on at least one of a user input and/or an event along the insertion trajectory related to the force data; and
record additional flagged points at predetermined time or a distance interval along the insertion trajectory; and
output the force data as graphical data.

64. The system of claim 62, wherein the processor is further configured to:
determine whether the insertion results in a navigation error; and
upon determining the navigation error, stop the insertion, record a location of the navigation error, and at least one of recommend and/or control a corrective action that includes retracting the at least one sensor to a flagged point adjacent to the location of the navigation error.

65. A system, comprising:
a catheter having one or more bendable segments and at least one sensor at a distal end thereof; and
a processor configured to:
control insertion of at least a portion of the catheter into a lumen along an insertion trajectory;
record a navigation parameter at a plurality of time-points and/or a plurality of positions along the insertion trajectory using the at least one sensor;
detect a navigation error;
display, on a display, at least one of the navigation parameter and/or a representation of the navigation parameter relative to a threshold value or the navigation parameter recorded at prior positions along the insertion trajectory; and
provide a corrective feature that includes one or more of: (a) an interactive user guide showing movement of the catheter to correct the navigation error, and/or (b) a flagged point corresponding to an insertion depth, with the flagged point including one or more of an error-free location or an error-prone location along the insertion trajectory.

66. The system of claim 65, wherein, upon detecting the navigation error, the processor controls at least one of: entering a relax mode, retracting the catheter the error-free location or the error-prone location, and re-inserting the catheter along a corrected insertion trajectory to one of error-free location or the error-prone location.
